(19) United States
(12) Patent Application Publication
(10) Pub. No.: US 2020/0399571 A1
(43) Pub. Date: Dec. 24, 2020

(54) CELL CULTURING DEVICE AND METHOD

(71) Applicant: BAR-ILAN UNIVERSITY, Ramat-Gan (IL)

(72) Inventors: Mordechai Deutsch, Olash (IL); Naomi Zurgil, Herzlia (IL); Elena Afrimzon, Petah Tikva (IL); Sergei Moshkov, Petah Tikva (IL); Orit Ravid-Hermesh, Ramat-Gan (IL); Yana Shafran, Petah Tikva (IL); Maria Sobolev, Kfar-Saba (IL)

(21) Appl. No.: 16/935,250

(22) Filed: Jul. 22, 2020

Related U.S. Application Data
(63) Continuation of application No. PCT/IL2019/050517, filed on Jan. 22, 2019.
(60) Provisional application No. 62/620,497, filed on Jan. 23, 2018.

(51) Int. Cl. C12M 1/32; C12M 1/12; C12M 1/00; C12N 5/00

(52) U.S. Cl. CPC C12M 23/12; C12M 25/14; C12N 2513/00; C12N 5/0075; C12N 5/0062; C12M 23/22

(57) ABSTRACT

A cell culturing device and method of using same are provided. Embodiments of the cell culturing device include a plate having at least one well with a through-hole formed at a bottom wall thereof and a hydrogel matrix disposed in the through hole. The cell culturing device can also include an optically transparent plate at the bottom of the through-hole.

### BACKGROUND

The present invention relates to a cell culturing device and to a method of using same for studying cells and 3D multicellular objects. More particularly, the present invention relates to a multi-well plate having through-holes at a bottom of each well with a hydrogel matrix embossed with or without sub-microliter wells disposed in the through-hole.

Numerous types of multi-well plates are commercially available for culturing cells and performing biological or chemical assays. Such multi-well plates are relatively easy and inexpensive to manufacture and provide the structural integrity necessary for manual or automated handling.

Multi-well plates are typically fabricated as an ordered array of individual wells each having sidewalls and a bottom so that liquid sample can be placed within each well. Multi-well plates can have a well count ranging from 4 to 1536 macro-wells.

The materials used to construct multi-well plates are selected based on the samples to be assayed and the analytical techniques to be used. Such materials are typically chemically inert to the components of the sample and can be impervious to radiation or heating.

Some uses of multi-well plates require a transparent well bottom for assaying samples using spectroscopic or microscopic techniques. Optically transparent and ultraviolet transparent bottomed multi-well plates are available commercially and are typically made from two different polymeric materials, one used for the sidewalls of the wells and another for the bottom walls of the wells.

Multi-well plates that have well bottoms made from glass are also known. Glass is advantageous in that it is chemically inert and is optically superior to polymers. Glass can be processed to provide a surface having extreme smoothness and very little background signal. Also glass is better for high resolution imaging of cells.

Although glass is optically superior to polymers, it is extremely difficult to produce multi-well plates from glass. One solution to the problem is to join a plastic upper portion forming the sidewalls of the wells with a flat transparent glass lower portion forming the bottom walls of the wells. One commonly employed method of joining a plastic upper plate and a glass lower plate to one another is to use an adhesive.

Although such hybrid plates are far better suited for spectroscopic or microscopic studies, they are more expensive to produce and can fail at the seam joining the two portions.

There remains a need for, and it would be highly advantageous to have, multi-well plates with transparent bottoms and micro chambers (e.g., micro-wells) suitable for culturing of single cells and cell aggregates.

### SUMMARY

According to one aspect of the present invention there is provided a cell culturing device comprising a multi-well plate having at least one macro-well with a through-hole formed at its bottom thereof and a hydrogel matrix disposed in the through-hole.

According to another aspect of the present invention there is provided a method of culturing one or more cell types comprising providing the cell culturing device described herein; seeding one or more cell types within the picoliter to microliter chamber; and subjecting the cell culturing device to conditions suitable for culturing one or more cell types.

According to another aspect of the present invention there is provided a method of manufacturing a culturing device comprising providing a plate having at least one well with a through-hole formed at a bottom wall thereof; filling the through-hole with a hydrogel and embossing at least one cell culturing chamber in the hydrogel.

According to another aspect of the present invention there is provided a device for cell culturing comprising a plate having at least one well with a through-hole formed at a bottom wall thereof and a hydrogel matrix disposed in the through hole. The device further includes a ring disposed on top of the hydrogel matrix, the ring being fillable with a gel including at least one ECM component.

According to an aspect of some embodiments of the teachings herein there is also provided a cell culturing device comprising: a) a plate having at least one well with a through-hole formed at a bottom wall thereof; and (b) a hydrogel matrix disposed in the through hole.

In some embodiments, the device further comprises: (c) at least one chamber formed in the hydrogel matrix.

In some embodiments, the through-hole is shaped so as to trap the hydrogel matrix therewithin.

In some embodiments, the hydrogel matrix extends into the at least one well.

In some embodiments, the device further comprises an optically transparent support positioned under the plate, wherein the hydrogel matrix disposed in the through-hole contacts a top surface of the support.

In some embodiments, the through hole is shaped as a truncated cone. In some such embodiments, the through-hole has a diameter ranging from 2-32 mm.

In some embodiments, an inner surface of the through hole includes at least one undercut region. In some such embodiments, the undercut has a depth of 0.5-2 mm.

In some embodiments, the inner surface of the through hole includes protrusions directed radially inward. In some such embodiments, the protrusions are 0.5-3.5 mm in length.

In some embodiments, the device comprises a plurality of picoliter to microliter chambers formed in the hydrogel matrix.

In some embodiments, the picoliter to microliter chamber are shaped as an inverted truncated pyramid.

In some embodiments of the device, each of the picoliter to microliter chambers has a volume ranging from 1-50 nanoliters.

In some embodiments, the device further comprises a ring positionable in the at least one well, the ring including a circumferential inner groove. In some such embodiments the ring is 2-32 mm in diameter.

In some embodiments, the device further comprises a double ring insert positionable in the at least one well, the double ring insert including a central opening defined by an inner ring of the double ring insert and a plurality of compartments defined between the inner ring and an outer ring of the double ring insert.

In some embodiments of the device, the double ring insert includes at least one circumferential groove within an inner wall of the inner ring.

According to an aspect of some embodiments of the teachings herein there is also provided a method of culturing one or more cell types comprising: a) providing a cell culturing device according to the teachings herein; b) seeding one or more cell types within the picoliter to microliter chamber; and c) subjecting the cell culturing device to conditions suitable for culturing the one or more cell types. In some embodiments of such a method, the chamber is formed within a plurality of compartments each being for seeding a cell type.

According to an aspect of some embodiments of the teachings herein there is also provided a method of manufacturing a culturing device comprising: a) providing a plate having at least one well with a through-hole formed at a bottom wall thereof; b) filling the through-hole with a hydrogel; and c) embossing at least one cell culturing chamber in the hydrogel.

In some embodiments, the method further comprises positioning a double ring insert within the well prior to (b).

In some embodiments of the method, the double ring insert includes a central opening defined by an inner ring of the double ring insert and a plurality of compartments defined between the inner ring and an outer ring of the double ring insert.

In some embodiments of the method, the double ring insert includes at least one circumferential groove within an inner wall of the inner ring for trapping the hydrogel.

According to an aspect of some embodiments of the teachings herein there is also provided a cell culturing device comprising: a) a plate having at least one well with a through-hole formed at a bottom wall thereof; b) a hydrogel matrix disposed in the through-hole; and c) a gel disposed on top of the hydrogel matrix.

In some embodiments of the cell culturing device, the gel includes at least one extracellular matrix (ECM) component. In some such embodiments, the gel includes at least one extracellular matrix (ECM) component is disposed within a ring including a continuous/segmented circumferential groove along an inner surface thereof for trapping the gel.

In some embodiments the gel including at least one extracellular matrix (ECM) component is disposed within a double ring insert positioned in the at least one well, the double ring insert including a central opening defined by an inner ring of the double ring insert and a plurality of compartments defined between the inner ring and an outer ring of the double ring insert. In some such embodiments, the double ring insert includes at least one circumferential groove within an inner wall of the inner ring for trapping the gel including at least one extracellular matrix (ECM) component.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B schematically illustrate an embodiment of the present device.

FIG. 2 schematically illustrates a hydrogel micro-chamber of an embodiment of the present device.

FIG. 3 schematically illustrates a macro-well through-hole having an indentation/undercut.

FIGS. 4A-E schematically illustrate fabrication of the hydrogel micro-chamber array in a macro-well filled with a hydrogel matrix.

FIG. 5 illustrates 6 and 24 well plates (images left and right respectively) having through-holes at a bottom of each macro-well and ear-like indents in the sidewall.

FIG. 6 illustrates ear-shaped undercuts/indents in a sidewall of the through-holes.

FIGS. 7A-B illustrate a microchamber stamping device according to embodiments of the present invention. FIG. 7A illustrates 6 and 24-array stamping devices (top and bottom images respectively), FIG. 7B illustrates a stamping device having a notch on each stamping protrusion.

FIG. 8 illustrates incubation of the culturing device prior to array stamping.

FIG. 9 illustrates hydrogel micro-chamber array (HMA) stamping using 6 and 24-array stamping devices (top and bottom images respectively).

FIG. 10 illustrates a top view of the formed array within the hydrogel filled through-hole (bottom image) corresponding to a schematic side view of the formed array (top).

FIG. 11 illustrates a 6 and 24-well plate with formed HMAs.

FIG. 12 illustrates formation HMAs surrounded with a slotted ring.

FIG. 13 illustrates an embodiment of the present culture devices suitable for co-culturing of two or more cell populations/types.

FIG. 14 illustrates culturing of three cell types in a single macro-well having two HMAs surrounded by a slotted ring.

FIG. 15 are graphs illustrating the growth ratio, relative growth ratio (in comparison to control) and % of the PI stained area in cultured MCF7 breast cancer spheroids following dose dependent drug treatment with Tamoxifen.

FIGS. 16-17 illustrate various views of one embodiment of an insert utilizable with the device of the present invention.

FIG. 18 illustrates the position of the insert of FIGS. 16-17 with respect to the stamped microchamber array.

FIG. 19 illustrates another embodiment of the insert of the present invention.

### DETAILED DESCRIPTION

The present invention is of embodiments of a cell culture device which can be used to culture cells or 3D multicellular objects. Specifically, embodiments of the present invention can be used to culture cells under conditions suitable for formation of 3D multicellular objects (e.g. spheroids) and to study cells/3D multicellular objects invasion capabilities, interactions between two or more cell populations and the affect of drugs on cells and 3D multicellular objects.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Multi-well plates having optically transparent bottoms are known in the art. Such plates are fabricated having optically transparent polymer or glass bottoms. Although such plates can be optically interrogated using microscopic or spectrographic techniques, the polymer-bottom plates are limited by the optical properties of the polymer used for the well bottom while the glass-bottom plates are limited by cost and integrity of the polymer-glass interface.

Previously filed patent applications to the present inventors disclosed a multi-well plate having a hydrogel matrix embossed with picoliter chambers. These patent applications and subsequent studies have shown that hydrogel-formed picoliter chambers are highly effective for generating and studying 3D multicellular objects such as spheroids.

While reducing the present invention to practice, the present inventors have set out to devise plates having hydrogel-embossed nano-liter to micro-liter chambers (e.g. wells) that are optically transparent and can be interrogated using microscopic or spectrographic techniques.

As is further described herein below and in the Examples section that follows, the present inventors have constructed multi-well plates having a hydrogel matrix embedded within a through-hole formed at the bottom wall of each well. The through-holes were designed to provide the required optical transparency for each well and each sub-microliter chamber formed therein as well as to maintain the hydrogel solution trapped within the hole during matrix solidification.

Thus, according to one aspect of the present invention there is provided a device for culturing cells.

As used herein, the term culturing refers to subjecting cells or 3D multicellular objects (e.g., spheroids—homogeneous or heterogeneous aggregates in which the cells retain tissue specific function) to conditions suitable for studying cells/3D multicellular objects. Such conditions can maintain viability of the cells or 3D multicellular objects and/or support replication, differentiation, motility and the like.

Examples of cells that can be cultured by the present device include, but are not limited to eukaryotic and prokaryotic cells. Examples of eukaryotic cells include human cells, animal cells and plant cells. The cells can be differentiated or non-differentiated, normal or cancerous, cell lines or primary cells from human and animal specimens. Examples include stem cells, cancer stem cells, circulating tumor cells, induced pluripotent stem cells, embryonic stem cells, normal adult cells, cancer cells, transformed cells and the like.

Human or animal cells can include normal cells such as hematopoietic cells, blood cells, cord blood cells, immune cells, nerve system cells, epithelial cells, endothelial cells, hepatocytes, and the like or pathogenic cells (e.g. tumor/cancer cells) from the categories of Carcinoma, Leukemia, Lymphoma, myeloma, Sarcoma, Central nervous system, Mesothelioma and the like.

Embodiments of the cell culturing device of the present invention can include a plate having at least one well (also referred to herein as macro-well) with a through-hole formed at a bottom wall thereof (‘floor’ of the well). The through-hole can be drilled/machined at the bottom wall following casting of the multi-well plate or alternatively, the multi-well plate can be cast with the through-hole (preformed plates having a through-hole).

The plate can be a multi-well plate having 4-96 or more macro-wells fabricated from a transparent or opaque polymer such as polystyrene, polypropylene or the like. Macro-well plate configurations that can be used as the initial design for embodiments of the present invention include, for example, COSTAR® Corning Incorporated 3516 (6 well plates), 3513 (12 well plate), 3524 (24 well plate), 3548 (48 well plate), 3595 (96 well plate) or Jet BIOFIL® TCP011004 (4 well plate), TCP011006 (6 well plate), TCP011012 (12 well plat), TCP011024 (24 well plate), TCP011048 (48 well plate), TCP011096 (96 well plate) or commercial plate with glass bottom such as Cellvis P06-14-0-N, P06-14-1-N, P06-14-1.5-N, P06-20-1-N, P06-20-1.5-N (6 Micro-well Glass Bottom Plates), P06-1.5H-N (6 well Glass Bottom Plates), P12-1.5H-N, P12-1.5P (12 well Glass Bottom Plates), P24-0-N, P24-1.5H-N, P24-1.5P (24 well Glass Bottom Plates), P96-0-N, P96-1-N, P96-1.5H-N, P96-1.5P (96 well glass bottom plates) for the insert version and others. The macro wells can be round (cylindrical), square or any shape suitable for use in multi-well plates with straight or tapering sides (e.g., tapering downward). Typical macro well dimensions can be 6-35 mm in diameter and 10-18 mm in well depth. The bottom wall of each well can be 1-2.5 mm in thickness (polymer) or 0.085-0.175 mm (glass).

The through-hole provided at the bottom of each macro well can be circular, square or any other shape. The through-hole can have straight sides, i.e., cylindrical in the case of a circular hole, or it can taper from bottom to top, i.e. conical in the case of a circular hole. The through-hole can include indentations or protrusions along a side wall thereof or undercuts at the bottom of the wall. Typical dimensions for the through-hole can be 3-31 mm in diameter and 0.6-2.6 mm in height. The indentations/undercuts or protrusions can be, for example, 0.4-100 mm³ in volume and the side walls of the though-hole can be textured (roughened).

Embodiments of the cell culturing device can include an optically transparent cover at the bottom of each well. Such a cover can be fabricated from glass (e.g., cover glass thickness no. 1 manufactured by Paul Marienfeld GmbH & Co. KG, Waldemar Knittel Glasbearbeitungs GmbH, Thermo Scientific, Menzel GmbH etc.) or an optically transparent polymer such as polystyrene, polypropylene or the like. The support plate can be glued or otherwise attached to the bottom of the multi-well plate. Commercial plates with glass bottom as described above (Cellvis) could be used as well.

The through-hole at the bottom of each macro well is filled with a hydrogel matrix composed of Agar or Alginate (natural carbohydrate from algae), Agarose (natural carbohydrate from seaweed) or synthetic polymers (e.g., poly (ethylene glycol) (PEG) and Pluronic®) or naturally derived proteins (e.g., collagen, gelatin, fibrin, fibronectin, laminin, tenascin, versican, elastin etc. (natural peptide from mammals), or glycosaminoglycan (heparin/heparin sulfate, chondroitin sulfate/dermatan sulfate, keratan sulfate and hyaluronic acid (natural carbohydrate from mammals) or a mixture of two or more hydrogels. An agarose hydrogel matrix can be composed of a 1-10% agarose [e.g., Low-melt agarose (LMA) from Cambrex Bio Science Rockland, Inc., Rockland, Me. USA].

Hydrogels are shape-retentive polymeric networks swollen with a high percentage of water (T. K. Merceron and S. V. Murphy, “Hydrogels for 3D Bioprinting Applications,” in Essentials of 3D Biofabrication and Translation, Elsevier, 2015, pp. 249-270.). A hydrogel can be composed of a naturally derived proteins or glycosaminoglycans (e.g., collagen, gelatin, fibrin and hyaluronic acid etc.), Alginate and Agar (natural carbohydrate from algae), Agarose or synthetic polymers such as polyethylene glycol (PEG) and Pluronic®.

These molecules can be mixed with cells and other bioactive factors or embedded on pre-cultured cells, in aqueous solution and then be manipulated to form an insoluble, cross-linked meshwork, resulting in a cell-laden hydrogel. Manipulation from the monomeric/un-cross-linked form to the polymeric/cross-linked form is accomplished by inducing physical or chemical bonding through environmental changes (such as pH, temperature, and ionic concentration), enzymatic initiation, or photo polymerization.

Hydrogels are an attractive medium for cell culturing because of their hydrophilicity and ability to encapsulate cells and bioactive molecules, thus mimicking many of the characteristics of natural ECM (J. Malda, J. Visser, F. P. Melchels, T. Jüngst, W. E. Hennink, W. J. A. Dhert, J. Groll, and D. W. Hutmacher, “25th Anniversary Article: Engineering Hydrogels for Biofabrication,” Adv. Mater., vol. 25, no. 36, pp. 5011-5028, September 2013.). In addition, they have good porosity for diffusion of oxygen, nutrients, and metabolites; can be processed under mild cell-friendly conditions; and produce little to no irritation, inflammation, or products of degradation (Fedorovich N E, Alblas J, Wijn J R, Hennink W E, Verbout A J, Dhert W J. Hydrogels as extracellular matrices for skeletal tissue engineering: state-of-the-art and novel application in organ printing. Tissue Eng. 2007; 13:1905-25.).

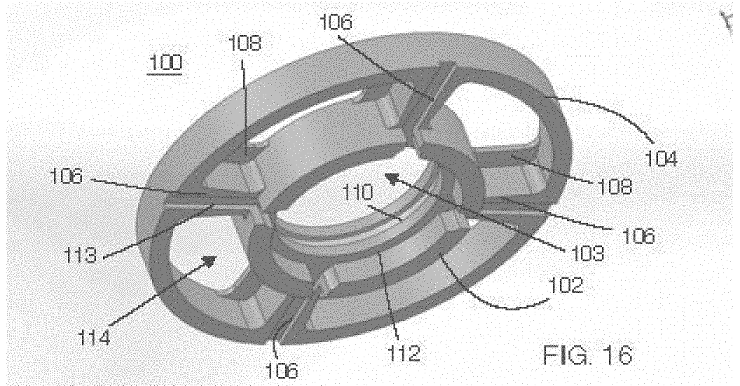

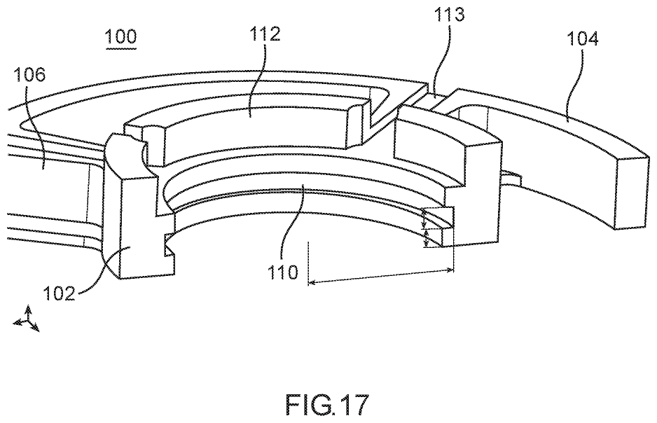

The hydrogel can be poured in-situ or it can be preformed (as a single or multiple well inserts). In the latter case, a template for one or more macro-wells can be used to fabricate one or more inserts that can then be fitted into respective through-holes of a multi-well plate or to a bottomless plate. Alternatively, an insert having a through hole and four or more circumferential wedge-shaped compartments can be placed within the macro well of a multi well plate and the hydrogel can be poured in-situ therethrough. The insert is shown in FIGS. 16-18 and is further described hereinbelow.

The indentations/protrusions/undercuts or textured side wall surface of the through-hole within the macro well bottom or a circumferential groove within the insert can trap and hold the hydrogel so as to prevent dislocation/movement thereof out of the through-hole following gelation.

The hydrogel matrix can be poured such that it fills the through-hole and optionally extend out of the through-hole and into the macro-well. In case of the insert, the hydrogel could fill the through-hole and extend to fill a circumferential channel within the insert to create a barrier between the wedge-shaped circumferential areas (for co-culturing 2-8 different types of cells in each one of the areas).

The hydrogel matrix includes a plurality of picoliter to microliter chambers formed therein via, for example, embossing using a dedicated tool.

The Examples section that follows describes a stamping/embossing device that can be used to fabricate the micro-wells in the top surface of the hydrogel matrix as well as method of using same for such fabrication.

The embossed chambers (also referred to herein as micro-chambers or micro-wells) provide the conditions and volume necessary for single cell culturing and formation of cell aggregates (e.g. 3D multicellular objects). Each matrix can include a single micro-chamber or an array of micro-chambers having 40-7400 micro-chambers (also referred to herein as a hydrogel array of micro-chambers or HMA). Depending on the macro-well size, the number of micro-chambers in an array and use, the volume of each micro-chamber can range between less than a nano-liter to hundreds of microliters (e.g., 0.5-50 nanoliters to 1-500 microliters). For example, a 96 well plate, HMC array of 40, 150 μm×150 μm square shaped truncated top flat inverted pyramid, micro well size bottom embossed on an area 3 mm in diameter each micro chamber has a volume of 16.59 nL.

The micro-wells can be of any shape suitable for culturing. One shape that can be used is an inverted truncated pyramid having a base (top) of 320-430 □m or larger and a truncated top (at bottom of micro-well) of 35-150 □m or larger. The height of the micro-well depends on the embossing pattern, height of the through-hole and whether or not the hydrogel matrix extends above the through-hole into the macro-well; a typical height can be 190 μm.

As is further described in the Examples section that follows, the present culturing device can be used to culture individual cells, to form 3D multicellular objects, to study the effect of compounds (e.g. drugs) on cells or 3D multicellular objects as well as to clone and differentiate stem cells.

The present device can also be used for invasion studies or to study the effects of one cell type on another.

In order to facilitate such studies, the present device can include a ring positionable in the macro-well on top of the hydrogel matrix. The ring can include a circumferential inner groove/slot. The ring can be fabricated from a polymer such as polystyrene and polypropylene with a diameter of 3-31 mm, a height of 3-10 mm and a thickness of 1-2 mm. The inner groove/slot can be 0.5-2 mm in depth (into the side wall of the ring). The slots can pass completely through the side wall of the ring or not. The groove/slot can be formed in the hydrogel matrix around the HMC array area during the embossing procedure. In the case of the insert, a circumferential and continuous/segmented groove at the inner part of the insert can be fabricated in order to trap the ECM and maintain it against the hydrogel.

For invasion studies, an ECM matrix in its soluble form is poured within the ring or within the insert or directly on top of the HMC array. The slot/groove in the ring or in the insert or in the hydrogel matrix around HMC array helps retain the ECM matrix in position against the HMC array, upon ECM gelation, thereby allowing contact between the cells and the ECM components in the ECM matrix.

Any size multi-well plate having any number of embossed micro-chambers can be used for invasion studies. For example, a 6 well plate with 1-3 embossed HMAs at each macro well can be used for an invasion assay. Cells can be loaded on the plate at a concentration of less than 5 cells/micro chamber (MC) by gently adding a cell suspension on top of the HMA allowing the cells to settle by gravity for 15 minutes. Next, aliquots of 50-500 μL fresh medium (total 2-4 mL for 6 well plates and total 1 ml for 24 well plates but not limited to) can be gently added to the rim of the macro-well plastic bottom alongside the hydrogel array and the plate can then be incubated at 37° C. for 24-72 hrs in order to form 3D multicellular objects.

Following 3D multicellular objects formation, the medium can be removed from the macro wells and ECM components (at least one component or a mixture of few ECM components e.g., collagen, gelatin, fibronectin, laminin, tenascin, versican, elastin, hyaluronic acid, heparan sulfate, chondroitin sulfate, keratan sulfate and others) can be poured gently on top of the 3D multicellular objects. Indicators/nanoparticles/beads for the measurement of enzyme activity/proteins/nucleic acids/exosomes and other factors which could be secreted from the spheroids can be mixed with the ECM gel or added to the micro chambers prior to gelation. Following gelation (for a collagen type I the plate is incubated at 37° C. for 1 hr) the ECM gel surrounds/covers the 3D multicellular objects and upon gelation is trapped by the slot formed in the ring or insert or in the hydrogel matrix.

The plates can then be incubated for several days and images can be acquired in order to follow movement of the whole 3D multicellular objects (shape change) or movement of single cells (moving out of the spheroid into the surrounding ECM).

Images can be acquired by any type of inverted microscope (Olympus, Nikon, Leica etc.) or any type of micro titer plate imaging unit (e.g., Celigo High Throughput Micro-Well Image Cytometer, JuLI stage, Cytation cell imaging multi mode reader, etc.) through the bottom glass covering the through-hole filled with the HMA.

Images can be analyzed manually by an expert or automatically by using image analysis software.

The present device can also be used to co-culture two or more cell types for cell-cell interaction studies.

A macro-well, having two embossed HMAs, can be used to study interactions between two cell populations sharing a single growth medium (covering both HMAs). Furthermore, and as described in the Examples section that follows, such a macro-well fitted with the ring can be used to study three different populations with the third seeded outside the ring and within the culture medium.

In the case of the insert, one to four (or more) different cell types could be seeded into the compartments created by the insert. Those compartments are localized around the HMA and enable studying of 5 different cell populations.

Embodiments of the present device can also enable seeding of cells at different areas of an array by providing removable barriers for seeding. Such barriers can be shaped like cookie cutters with the shape and size depending on the region of the HMA seeded. For example, a square shape 12×12 mm embossed area of HMC array is divided to 3 separated subareas having a triangle shape 4×12 mm each. Separation is performed by using plastic partitions. Other shapes could be designed as well, for example a circle divided into 4-8 sectors or segments. After loading the cells into the different regions, ECM is added into each region, the plastic barriers are removed and direct interaction between two (or more) cell types (e.g. invasion of cancer cells into stromal cells) is enabled, allowing co-cultured cells in adjacent areas to interact and/or migrate freely.

Embodiments of the present device can be used to retrieve one and more 3D multicellular objects according to its phenotype i.e. response to drugs, metabolic parameters, immunostaining, invasive capacity, etc by identifying phenotypes of each of the 3D multicellular objects using the HMC array and imaging techniques. Retrieved 3D multicellular objects can be further characterized using molecular and biochemical approaches.

The HMC array of the present invention enables one to monitor individual 3D multicellular objects without risk of 3D multicellular object dislocation throughout drug treatment, staining and other manipulations and to specifically retrieve individual 3D multicellular objects.

Embodiments of the present invention also enable retrieval and enrichment of specific cells. For example, when performing an invasion assay, cells invade from a 3D multicellular object into the surrounded ECM. Cells displaying such an invasive phenotype could be further analyzed using biochemical and molecular approaches.

3D multicellular objects can be retrieved from the HMC array used for an invasion assay leaving behind the invasive cells in the ECM. The cells can then be separated from the ECM for examination or for enrichment (loaded second time into the HMC array, spheroid creation, and committing invasion into ECM and retrieved, all this for several rounds).

Thus, the present invention provides a culturing device that can be used to seed individual cells, study cell and aggregate development and invasiveness, study the effect of drugs on individual cells and aggregates, isolate cells and 3D multicellular objects of a specific phenotype and identify and isolate cells having an invasive phenotype as well as study cell-cell interactions.

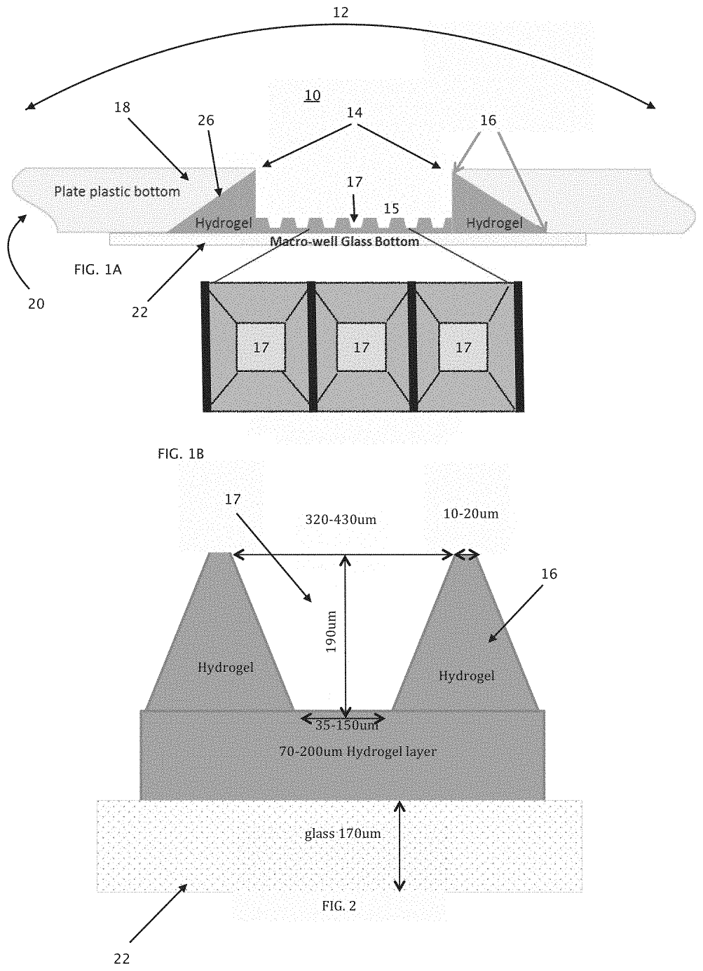

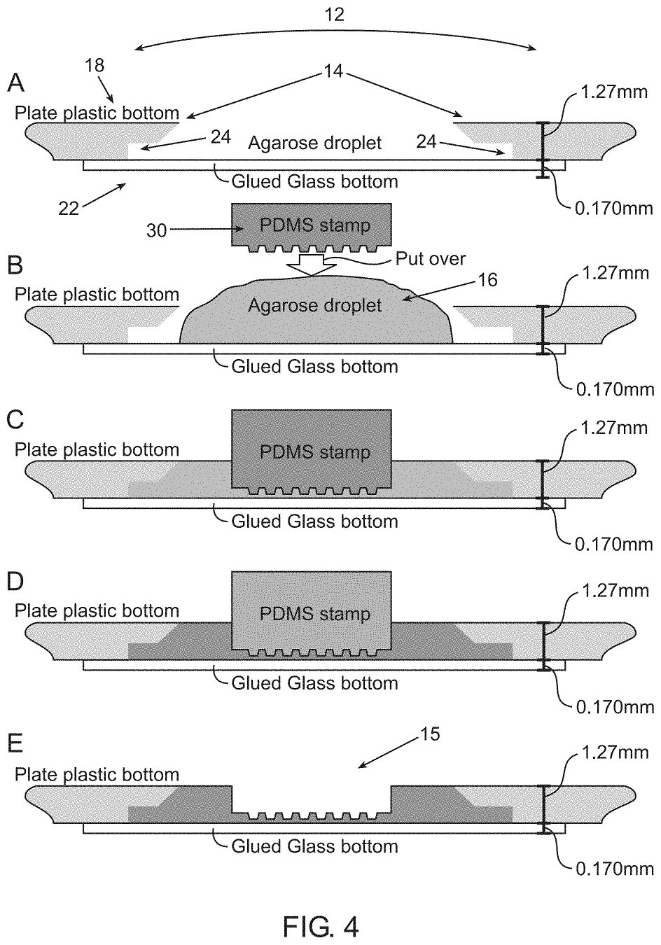

An embodiment of the present device, referred to herein as device 10 is shown in FIG. 1A to FIG. 3. Fabrication of device 10 is shown in FIG. 4.

FIG. 1A is a side schematic of a single macro-well 12 having a through-hole 14 filled with a hydrogel matrix 16 formed with a hydrogel matrix array 15 having a plurality of micro-chambers 17. FIG. 1B is a top view schematic of three micro-chambers 17 formed in hydrogel matrix 16.

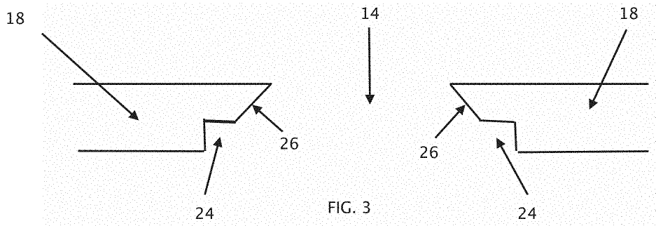

As is shown in these Figures, these embodiments of device 10 include a conically-shaped through-hole 14 through the plastic bottom 18 of plate 20. Through-hole is filled with hydrogel 16 which is embossed with a plurality of micro-chambers 17, each shaped as an inverted truncated pyramid. Typical dimensions for each micro-chamber 17 are shown in FIG. 2. A support 22 made of optically transparent material such as glass is attached at a bottom of through-hole 14.

FIG. 3 illustrates a through-hole 14 with an undercut/indentation 24 for trapping hydrogel matrix 16 within through-hole 14.

Undercut/indentation 24 can be provided along the circumference of through-hole 14 at, for example, a bottom end thereof (as is shown in FIG. 3) or at discrete regions of sidewall 26.

FIGS. 4A-E illustrate formation of HMA 15 using a stamping/embossing device 30.

FIG. 4A illustrates macro-well 12 with formed through-hole 14 and support 22 positioned thereunder.

A liquid agarose droplet is positioned on top of support 22 within through-hole 14 (FIG. 4B). A stamping/embossing device 30 is pushed into the agarose to form micro-chambers 17 and spread the liquid agarose within through-hole 16 such that it is trapped under indentations 24 (FIG. 4C). When the agarose sets after gelation (FIG. 4D), stamping/embossing device 30 is pulled out to leave behind HMA 15 (FIG. 4E).

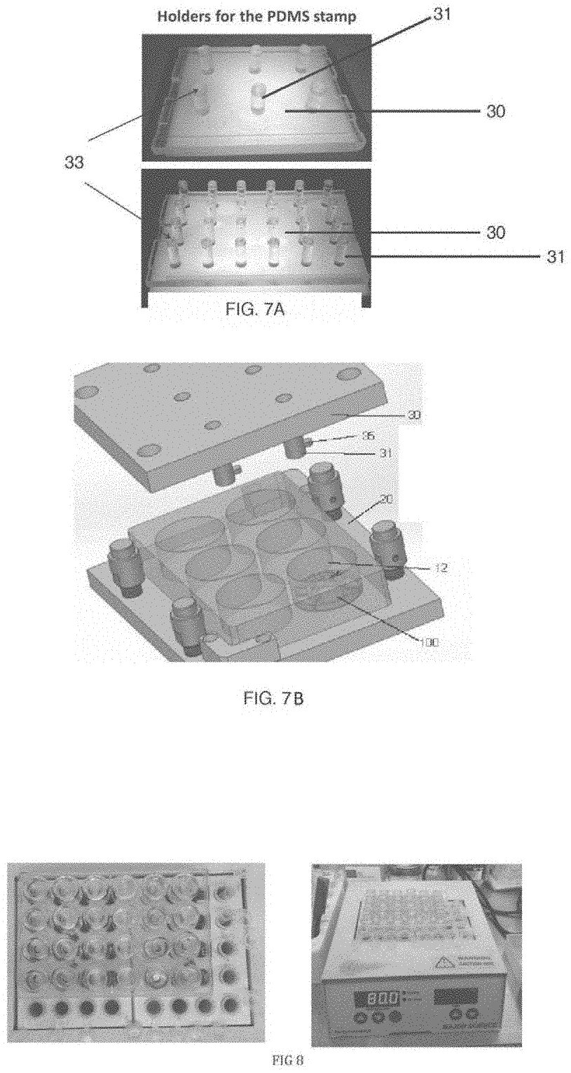

A stamping/embossing device 30 (also referred to herein as “PDMS stamp”) is shown in FIG. 7A. Device 30 includes a plurality of stalks/protrusions 31 each carrying microchamber array-forming template 33 on the distal face thereof. FIG. 7B illustrates device 30 in which the stamping protrusions 31 include a notch 35. Notch 35 forms a channel alongside the stamped microchamber array. The channel enables provision and removal of liquids (e.g. growth media) without disturbing/dislocating the cells/spheroids grown in the array. Notch 35 is configured to generate a channel that begins about 2 mm above the level of the array in order to avoid entrance of cells into the channel when the cells are loaded onto the array.

In order to avoid reshaping (texturing/channeling) the bottom wall of the macrowell, an insert having the general shape of the macrowell (e.g., spherical for sphere macrowells, square for square macrowells) and the structural components which prevent the hydrogel from shifting/dislocating can be utilized. The insert can include slots/channels to prevent the separation of the ECM from the hydrogel and several compartments for simultaneous culturing several cell types. The insert can be positioned within the macrowell and the hydrogel can be poured therethrough.

FIGS. 16-18 illustrate one embodiments of an in-well insert which is referred to herein as insert 100.

Insert 100 can be formed as a double ring with an inner ring 102 attached to an outer ring 104 via spokes 106. Inner ring 102 defines a central opening 103, while outer ring 104 defines compartments 114 (divided off via spokes 106).

Inner ring 102 can be 3-31 mm in diameter and 3-10 mm in height, while outer ring 104 can be 6-35 mm in diameter and 1-10 mm in height. Insert 100 further includes ‘wings’ 108 extending from spokes 106 for trapping the gel which could be poured in compartments 114.

A continuous or segmented circumferential groove 110 in inner ring 102 can be used to prevent detachment of the ECM while a second circumferential groove 112 (continuous or segmented) can be used to prevent hydrogel displacement. Circumferential groove 110 could be 0.3-1 mm depth and 1-2 mm height and its length depends on the ring circumference/diameter. Second circumferential groove 112 could be 0.3-2 mm depth and 1-3 mm height and its length depends on the ring circumference/diameter. Segmental circumferential grooves length sum could be approximately third to quarter of circumferential length.

Channels 113 at the bottom of each spoke 106 are fillable with hydrogel (when poured through insert 100) to create a barrier between compartments 114 (four shown, 2-8 or more are possible).

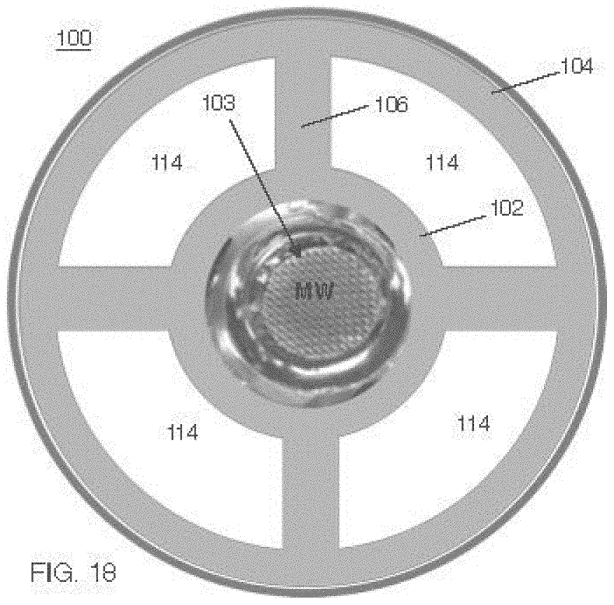

FIG. 18 illustrates the position of insert 100 with central opening 103 positioned over the macrowell (MW) with embossed microwells and compartments 114 surrounding the macrowell.

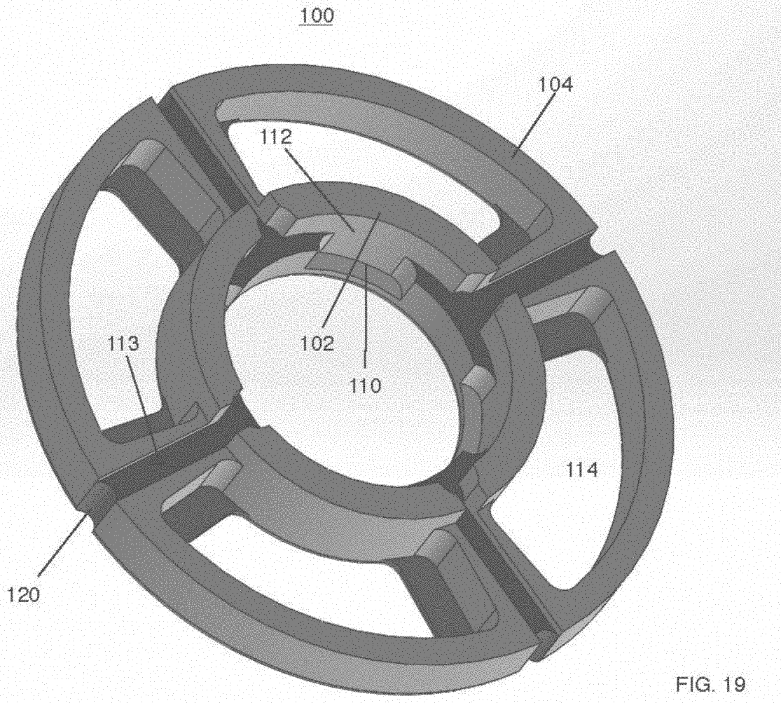

FIG. 19 illustrates an embodiment of insert 100 that includes semicircular indents 120 (half drills) on the outer edge of outer ring 104 and a segmented groove/slot 110 in the inner edge of inner ring 104. Indents 120 allow air to escape from channels 113 when the hydrogel is poured thereby facilitating complete filling of channels 113 with the hydrogel.

As used herein the term “about” refers to □10%.

Additional objects, advantages, and novel features of the present invention will become apparent to one ordinarily skilled in the art upon examination of the following examples, which are not intended to be limiting.

### EXAMPLES

Reference is now made to the following examples, which together with the above descriptions, illustrate the invention in a non limiting fashion.

### Example 1

### Fabrication of a Cell Culturing Plate with Through-Hole

Cell culturing plates having 6, 12, 24, 48 or 96 wells (also referred to herein as macro wells), with dimensions of about 127.8 mm×85.5 mm were modified in order to make the present device.

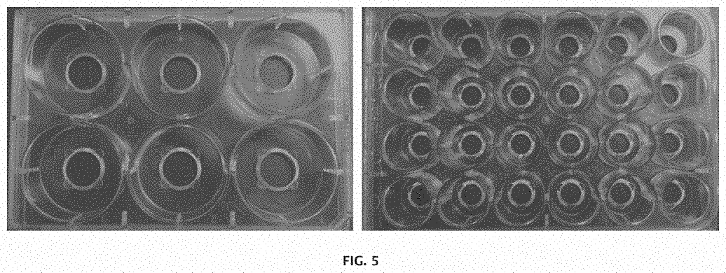

A conical aperture (through-hole) was cut in the middle of the plastic bottom wall of each macro well at an angle of 45° (FIG. 5). The conical-shaped through-hole was configured in order to prevent hydrogel vertical movements (floating). Additional through-hole elements such as undercuts (further described hereinbelow) were also used to avoid horizontal hydrogel movement and to further enhance hydrogel trapping within the through-hole.

The bottom diameter was selected such that at least a 2 mm ring (or wider) remained at the circumference of each macro well in order to enable gluing of a glass cover to the bottom of the plate. Depending on the plates used, the diameter of the through hole tapered from 6-31 mm at the bottom to 4-28 mm at the top. The depth of the through-hole depends on the thickness of the bottom wall of the macro well and can be, for example, 1-2.5 mm.

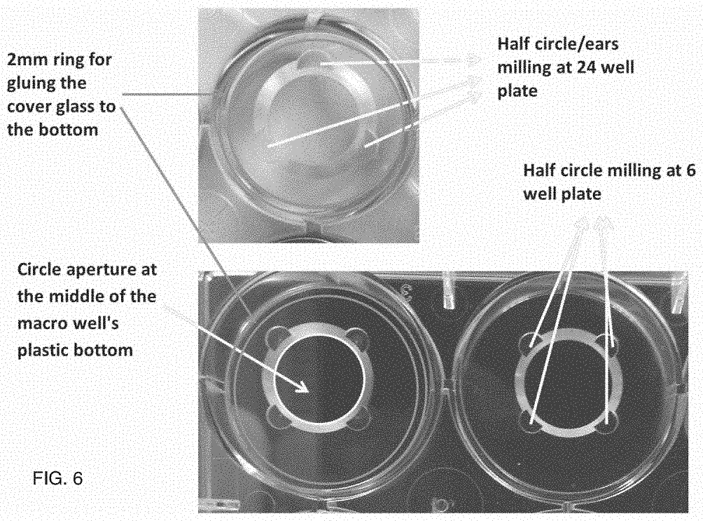

Several (3-4) undercuts were machined into the sides of the through-holes at a depth that is half of the bottom wall thickness (1.27 mm/2=0.635 mm at the 24 well plate and 1.8 mm/2=0.9 mm at the 6 well plate). FIG. 6 is a top view of the ear-shaped undercuts showing the through-hole and 2 mm rim/ring (for gluing of a cover glass).

An optically transparent support (glass plate) was adhered to the bottom of the macro well plate using polydimethylsiloxane (PDMS) and further glued at the glass edges by Norland Optical Adhesive (NOA) glued to the bottom of the macro well plate using polydimethylsiloxane (PDMS) or Norland Optical Adhesive (NOA). The ready plate was detoxified by abundant water washing to remove excess glue monomers.

### Example 2

### Fabrication of the Micro Chambers

A hydrogel matrix poured into the through-holes was embossed with a plurality of micro Chambers (also referred to herein as micro or sub micro wells) using a stamp.

### Stamping/Embossing Device

PDMS stamp heads were glued to cylinders fabricated from Plexiglas at different diameters. The other ends of the cylinders were glued to a Plexiglas holder. For a 6 well plate, the cylinder was 10 mm in diameter and for a 24 well plate, the cylinder was 6 mm in diameter (FIG. 7A, top and bottom images respectively). The PDMS stamp heads were sized accordingly.

The Plexiglas holder was sized in order to fit the upper opening of the macro well plate (like a lid/cover). When the holder is fitted into the macro well plate, the cylinders extend into the middle of each macro well. The length of each cylinder including the PDMS stamp head depended on the commercial plate chosen and its dimensions. The length of the cylinder with PDMS stamp is designed to leave a gap between the bottom of the micro-chambers and the glass plate, this gap is filled with the hydrogel matrix as shown in FIG. 2; 70-200 μm hydrogel layer.

An alternative stamping/embossing device fabricated from a metal such as stainless steel (in place of the Plexiglas) was also used. Use of a metal cylinder and optionally a metal stamp head (in place of the PDMS) enables use of a temperature controller to heat/cool the stamping device.

### Micro Chamber Fabrication

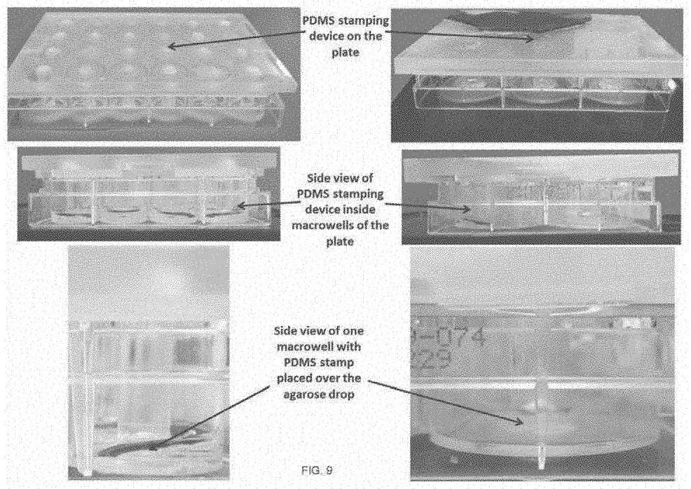

A small drop (±70 ul for 24 well plates and ±400 ul for 6 well plates) of Low Melting Agarose solution (6%) pre-warmed to 65-70° C. was symmetrically dripped into the through-hole on the surface of the glass bottom preheated to 80° C. on a dry bath (FIG. 8).

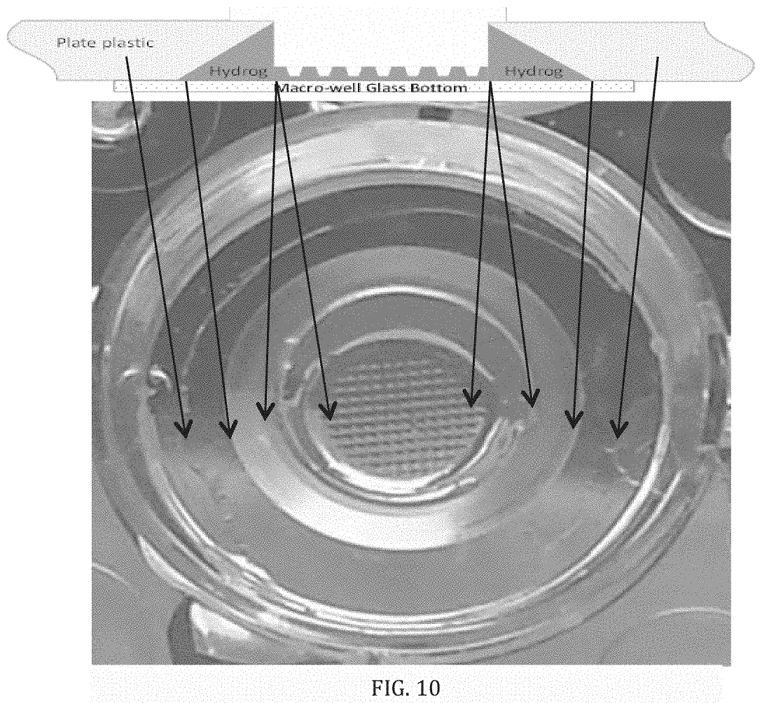

A pre-heated PDMS stamping device was then gently placed over the agarose drops as shown in FIG. 9.

The assembly was incubated at room temperature (RT) for 5-10 minutes for pre-gelling and pre-cooling, followed by 20 minutes incubation at 4° C. until full agarose gelation.

The stamping device was then peeled off, leaving the agarose gel patterned with micro-chambers (MCs).

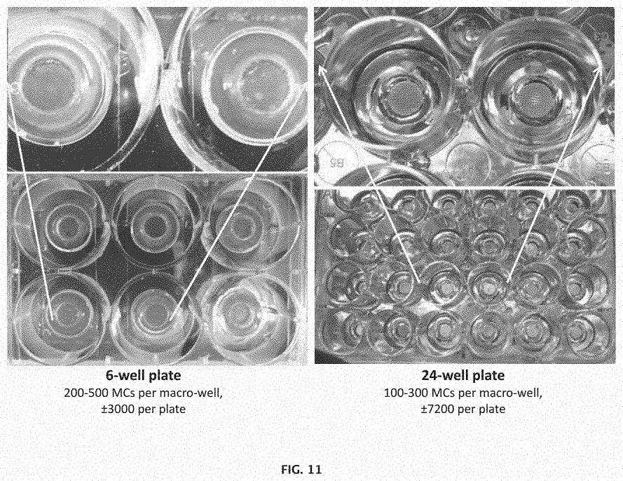

A top view of the formed MCs is shown in FIG. 10 with reference to a schematic side view showing the various regions of each macro well and MC.

The culturing plate comprising MCs was UV sterilized. The macro-wells were then filled with sterile phosphate buffered saline (PBS). The fully prepared plate was covered with Parafilm and stored at 4° C. in humidified atmosphere.

### Example 3

### Fabrication of Culturing Plates for Invasion Assays

Invasion assays utilize ECM components to identify cells capable of invasiveness. A plastic insert ring with a circumferential is positioned in the macro well at the junction between the plastic bottom of the macro well and the hydrogel. This insert can be held by pressure or glued to the plastic sides of the macro well or to the glass bottom. The insert surrounds the hydrogel array structure and a slot (prefabricated or fabricated post ring placement) traps ECM components poured over the hydrogel array in order to prevent floating/movement/separation of the ECM from the hydrogel structure when culturing medium is added to the macro well.

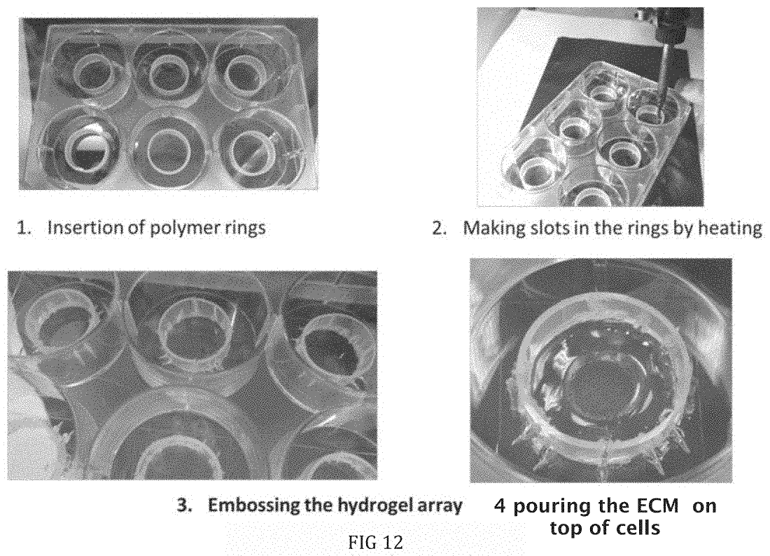

FIG. 12 illustrates fabrication of an invasion assay plate. The polymer ring is positioned in the macro well (1) and is glued to the well sides. If not prefabricated with a slot, each ring is then slotted using a soldering gun (2). A hydrogel is then poured into each macro well and embossed to create HMC array (3) and once gelled (and populated with cells), an ECM gel is poured over the cells (within the ring) and the plate is ready for invasion assaying (4).

### Example 4

### Fabrication of Culturing Plates for Co-Culturing Assays

Co-culturing plates were fabricated by embossing two or more MC array in a single through-hole filled with hydrogel. Embossing was effected using a PDMS stamping device having two side-by-side stamps.

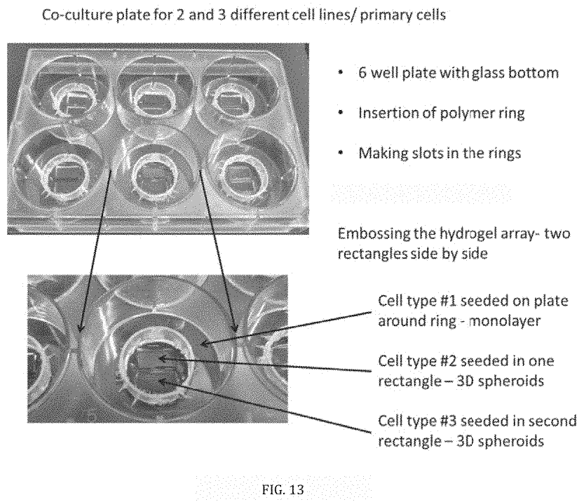

FIG. 13 illustrates a macro well having two side-by-side rectangular MC arrays with a ring insert positioned around the arrays for a co-culturing invasion assay. Such a plate enables seeding of three different types of cells—a first around the ring, a second in the first array and a third in the second array. This enables to measure interaction (through the shared culture medium) between three types of cells. This two array plate can also be used without the ring in two cell co-culturing assays.

### Example 5

### Plates with Cells

Cells are loaded onto the plates at different concentrations depending on the assay/experiment and the plate and MC dimensions.

For single cell experiments and self-renewal/clone formation experiments it is important that only one cell is seeded within each MC and as such, smaller MC are typically used (35×35 μm).

For 3D multicellular objects production experiments, more than one cell is seeded in each MC. The number of cells loaded on each MC depends on the size of 3D multicellular objects needed in the experiment. Usually 5-100 cells or more are seeded into each MC (90×90-150×150 μm or larger). The concentration of cells needed to be loaded is calculated based on: 1) number of cells/MC; 2) the volume of loaded medium—the volume depends on the area and shape of the MC array embossed. For example: For the round 10 mm diameter array on 6 well plate a volume of 60 μl is needed for 500 MC. For 1 cell/MC, 500 cells/60 μl are needed therefore a concentration of 8333 cells/ml is used for loading.

Plates with cells/formed spheroids can be maintained for days and transported to an end user.

### Example 6

### Invasion Assay Protocol

Mature 3D spheroids are prepared on 6-well invasion plates. The plate is cooled on ice for 10 minutes and the 3D spheroids are overlaid along with a collagen type I solution (3 mg/ml) (Cultrex, Rat Collagen I) mixed with DQ Gelatin FITC-conjugated substrate, and incubated at 37° C. for 1 h to reach full gelation.

DQ Gelatin FITC-conjugated substrate is a specific heavily fluorescein-labeled non-fluorescent gelatin substrate, enzymatically cleaved effectively by MMP-2 and MMP-9, to yield highly fluorescent peptides. Product fluorescence intensity (FI) reflects the MMP enzymatic activity level.

Invasion of spheroids is analyzed by real-time monitoring of cell clusters and individual invading cells which escape the sphere arrangement using an inverted microscope (further described below). Images will be acquired at 6 h intervals for 48 h, totaling 8 acquisitions. At the end of the experiment, spheroids and invading cells will be stained in situ for markers, fixed within the HMCA (hydrogel micro chamber array), and stained for intracellular markers in order to characterize the phenotype of invading cells vs. cells in spheroid body.

Parameters that can be extracted at each time point can include: (a) number of invading cells which escaped sphere margin/boundary indicated at time 0; (b) total invasion area (number of pixels); (c) maximal distance of invasion; (d) number of invading cells separated from connected invasion area and (e) FI of DQ Gelatin (MMP activity).

Based on the above parameters, spheroids can be classified either as highly invasive or low/non-invasive cell clusters. Corresponding morphometric and fluorescent readouts of all objects (spheroids and cells) will be utilized to define the invasive phenotypes.

A fully motorized, wide field inverted microscope with auto-focusing system and focus-map ability (Nikon, Olympus and Leica etc.) can be used to automatically acquire images at pre-defined time intervals from a series of regions on the HMCA (hydrogel micro chamber array). Each set of acquisitions will begin with the bright field image, followed by several fluorescent images, one for each fluorescent probe, taken at different preset time points.

Several algorithms have been developed for spheroid detection and morphometric parameter evaluation using bright field images and FI analysis. Cells/Spheroids can be automatically defined as regions of interest (ROIs) by modified Sobel edge detection and morphological operation, and their sectional area can be outlined on the bright field image. For each fluorescent wide field image, ROIs can be determined by mapping those outlines on the interrogated fluorescent field image. Following background subtraction and thresholding, two parameters can be automatically extracted: the mean FI value obtained for each ROI (mean FI of all pixels that are within the threshold borders) and the area fraction of the fluorescent signal.

Based on the above analysis scheme a set of algorithms for rapid multi-parametric processing and analysis of invasion assay and drug response can be developed. This algorithm set can include (a) an automatic segmentation algorithm of cells/spheres in their MCs, and individual invading cells, (b) object feature extraction: size, shape, texture and spatial location. (c) extraction of FI parameters for each object, for each fluorescent marker, (d) a complete framework for multi-parametric analysis of fluorescent image data and its corresponding bright field images for each measured object, yielding a full dataset that contains all the measured parameters related to each object at each time point, as well as changes in these parameters during the course of the experiment, (e) cluster analysis for classification of highly invasive vs. noninvasive cells/spheroids, (f) definition of different cell phenotypes, using supervised classification, based on the above classification (see e) and dataset (see d) and (g) machine learning analysis to predict invasion potential of spheroids enriched with invasive cell phenotypes, as well as their response to treatment.

### Example 7

### Co-Culturing of PrCSC Spheroids, Prostate Cancer-Associated Fibroblasts (PCAFs) and Inflammatory Cells

Major components of the cancer stroma have been shown to support tumor behavior, regulate cancer cells, maintain and significantly impact resistance to therapy (Shiao S L, Chu G C-Y, Chung L W K. Regulation of prostate cancer progression by the tumor microenvironment. Cancer Lett [Internet]. Elsevier Ireland Ltd; 2016; 380: 340-8.), (Eder T, Weber A, Neuwirt H, Grunbacher G, Ploner C, Klocker H, Sampson N, Eder I E. Cancer-Associated Fibroblasts Modify the Response of Prostate Cancer Cells to Androgen and Anti-Androgens in Three-Dimensional Spheroid Culture. Int J Mol Sci. 2016; 17: 1-15.) and (Maolake A, Izumi K, Shigehara K, Natsagdorj A. Tumor-associated macrophages promote prostate migration through activation of the CCL22-CCR4 axis cancer. 2017; 8: 9739-51.).

Prostate Cancer stem cells (PrCSC) spheroids can be co-cultured in Co-culture plates (CCPs) alongside PCAFs and inflammatory cells (macrophage-like cells).

M2 macrophage cells were identified histologically in prostate cancer (PrC) tissue at various stages of disease (Thapa D, Ghosh R. Chronic inflammatory mediators enhance prostate cancer development and progression. Biochem Pharmacol [Internet]. 2015 [cited 2015 Nov. 1]; 94: 53-62.) and (Lanciotti M, Masieri L, Raspollini M R, Minervini A, Mari A, Comito G, Giannoni E, Carini M, Chiarugi P, Semi S. The role of M1 and M2 macrophages in prostate cancer in relation to extracapsular tumor extension and biochemical recurrence after radical prostatectomy. Biomed Res Int. 2014; 2014:486798.). They promote PrC survival, adhesion, invasion and metastasis and mutually support tumor progression by modulating levels of cytokines, growth factors and reactive oxygen species in PrC microenvironment (Thapa D, Ghosh R. Chronic inflammatory mediators enhance prostate cancer development and progression. Biochem Pharmacol [Internet]. 2015 [cited 2015 Nov. 1]; 94: 53-62.), (Pinato D J. Cancer-related inflammation: an emerging prognostic domain in metastatic castration-resistant prostate carcinoma. Cancer [Internet]. 2014 [cited 2015 Nov. 1]; 120: 3272-4.) and (Shiao S L, Chu G C-Y, Chung L W K. Regulation of prostate cancer progression by the tumor microenvironment. Cancer Lett [Internet]. Elsevier Ireland Ltd; 2016; 380: 340-8.). It has been previously demonstrated that U937-M cells promote PrC proliferation and invasion (Lindholm P F, Lu Y, Adley B P, Vladislav T, Jovanovic B, Sivapurapu N, Yang X J, Kajdacsy-Balla A. Role of monocyte-lineage cells in prostate cancer cell invasion and tissue factor expression. Prostate [Internet]. 2010 [cited 2015 Nov. 1]; 70: 1672-82.) and serve as a niche to support CSC growth (Maolake A, Izumi K, Shigehara K, Natsagdorj A. Tumor-associated macrophages promote prostate migration through activation of the CCL22-CCR4 axis cancer. 2017; 8: 9739-51.) and (Lau E Y-T, Ho N P-Y, Lee T K-W. Cancer Stem Cells and Their Microenvironment: Biology and Therapeutic Implications. Stem Cells Int [Internet]. 2017; 2017: 1-11.).

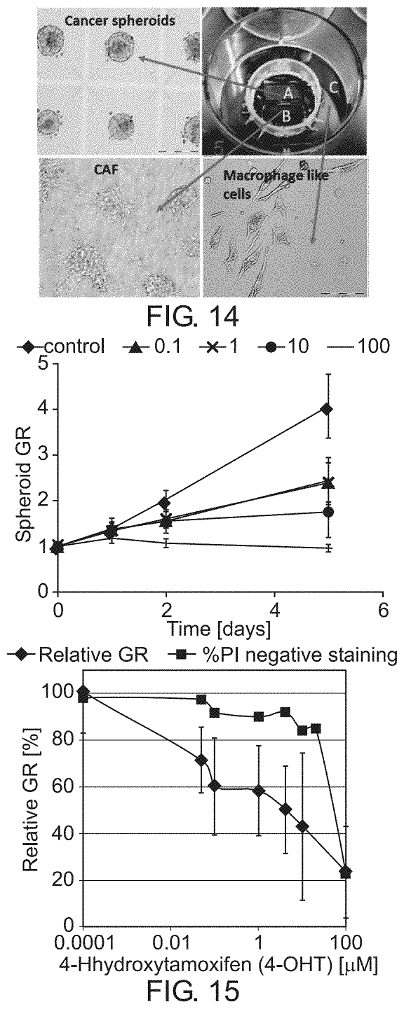

Floating U937 cells can be treated with phorbol 12-myristate 13-acetate (PMA) (10 ng-1 μg/mL) to obtain adherent macrophage-like cells (U937-M). Then, M2-type can be selected by culturing in conditioned medium of the PrC cells (Maolake A, Izumi K, Shigehara K, Natsagdorj A. Tumor-associated macrophages promote prostate migration through activation of the CCL22 CCR4 axis cancer. 2017; 8: 9739-51.), and grown in the outer border of the macro well, on the plastic bottom around the HMC array (See, FIG. 14). M-subtypes can be identified by staining for CCR7 and CD163 (M1 and M2, respectively).

Prostate cancer-associated fibroblasts (PrCAFs) modulate remodeling of the ECM (Tuxhorn J A, Ayala G E, Smith M J, Smith V C, Dang T D, Rowley D R. Reactive stroma in human prostate cancer: induction of myofibroblast phenotype and extracellular matrix remodeling. Clin Cancer Res. 2002; 8: 2912-23.), tumor proliferation (Shaw A, Gipp J, Bushman W. The Sonic Hedgehog pathway stimulates prostate tumor growth by paracrine signaling and recapitulates embryonic gene expression in tumor myofibroblasts. Oncogene. Macmillan Publishers Limited; 2009; 28: 4480-90.) and (Schauer I G, Rowley D R. The functional role of reactive stroma in benign prostatic hyperplasia. Differentiation. 2011; 82: 200-10.), angiogenesis (Webber J P, Spary L K, Sanders A J, Chowdhury R, Jiang W G, Steadman R, Wymant J, Jones A T, Kynaston H, Mason M D, Tabi Z, Clayton A. Differentiation of tumour-promoting stromal myofibroblasts by cancer exosomes. Oncogene 2015; 34: 290-302.) and drug sensitivity in PrC cells (Cheteh E H, Augsten M, Rundqvist H, Bianchi J, Same V, Egevad L, Bykov V J, Ostman A, Wiman K G. Human cancer-associated fibroblasts enhance glutathione levels and antagonize drug-induced prostate cancer cell death. Cell Death Dis [Internet]. Nature Publishing Group; 2017; 8: e2848.). The hTERT PF179T CAF (ATCC® CRL-3290TM) immortalized cells, present an appropriate stromal model for a PrC study (Madar S, Brosh R, Buganim Y, Ezra O, Goldstein I, Solomon H, Kogan I, Goldfinger N, Klocker H, Rotter V. Modulated expression of WFDC1 during carcinogenesis and cellular senescence. Carcinogenesis. 2009; 30: 20-7.). These PCAFs can be grown as spheroids within the hydrogel micro chambers since stromal cells in 3D configuration trigger PrC cells phenotypes to become more invasive (Windus L C, Glover T T, Avery V M. Bone-stromal cells up-regulate tumorigenic markers in a tumour-stromal 3D model of prostate cancer. Mol Cancer [Internet]. Molecular Cancer; 2013; 12: 112.).

Culture conditions for sustaining all cell populations within different compartments in the CCP under a mutual environment can be established including medium components, incubation and culturing time, initial cell density, ratio of stromal cells to tumor cells, cell seeding sequence (simultaneous/sequential seeding), medium exchange and duration of cell growth.

### Example 9

### Drug Screening

The cytotoxic potential and cell growth inhibition effect of tested drugs can be evaluated using the hydrogel micro chamber plates described herein.

A cell suspension can be loaded on the top of the HMA as described above and fresh cell medium can then be added. Individual cells or formed spheroids can be tested. A tested drug or drug candidate can be added to the cell medium of the plate wells at different concentrations, at different time points and for different incubation periods (different dosage and time of drug exposure).

Cells/spheroids can be imaged and measured before exposure to the tested drugs, and at different time points following addition of the drug (hours-days) and compared to non-treated cells/spheroids cultured under the same conditions. An exclusion test of cell viability can then be performed (using a fluorescent dye such as propidium iodide (PI) or a colorimetric dye such as trypan blue).

Several parameters can be evaluated including:

(i) The growth ratio (the ratio between spheroid sectional areas or between calculated spheroid volumes at two time points of individual spheroids)—this parameter is tested at every time point of the experiment.

(ii) IC50 value—the drug concentration that inhibits the spheroid growth by 50%.

(iii) The stained area of the spheroid (presenting dead cells)—this parameter is tested at the end point of the experiment only.

Using the above protocol, the effects of 4-hydroxytamoxifen (4-OHT) on MCF7 breast cancer spheroid growth was tested. MCF7 spheroids were generated and grown for 48 h in 24 well HMA-based imaging plate. Different concentrations of 4-OHT (0-100 μM) were added to the plate wells at different time periods (1-5 days). On the fifth day, the spheroids were stained with PI (2.5 μg/ml). The experiment was performed in triplicates; the growth ratio, relative growth ratio (in comparison to control) and % of the PI stained area were measured and are shown in the graphs of FIG. 15.

### Example 10

### Retrieval, Enrichment and Molecular Analysis of PrTMICs

Prostate cancer stem cells (PrCSCs) and Prostate Tumor metastatic initiating cells (PrTMICs) are important for understanding Prostate cancer (PrC), metastasis and development of efficacious therapies for eliminating this phenotype.

Embodiments of the present HMC array can be used to retrieve a PrTMIC population and to enrich and expand the TMIC phenotype.

Since both PrCSC spheroids and invaded cells are embedded within the hydrogel layer, recovery of pure TMIC populations can be effected using a two-step protocol in order to separate and retrieve the pools of PrC spheroids and potentially-metastatic invading cells. Relatively large structures of spheroids of interest can be manually picked up using a micromanipulator with a capillary tip, leaving the invaded cell pool within the hydrogel. This technique has been successfully used to retrieve clones encapsulated within collagen gel (Guan Z, Jia S, Zhu Z, Zhang M, Yang C J. Facile and Rapid Generation of Large-Scale Microcollagen Gel Array for Long-Term Single-Cell 3D Culture and Cell Proliferation Heterogeneity Analysis. Anal Chem. 2014; 86: 2789-97. doi: 10.1021/ac500088m.). Isolated PrC spheroids can then be used to further analyze PrCSC renewal capacity and gene analysis. Enzymatic degradation of agarose and ECM can be used to recover invasive cell populations (Bates M. Three-Dimensional Mammalian Cell Culture Using Hydrogel Filled Scaffold. TISSUE Eng [Internet]. 2013 [cited 2015 Nov. 2. Available from www.msoe.edu/servlet/JiveServlet/downloadBody/4262-102-1-5486/Paper_MBates.pdf) by adding β-Agarase (agarose 4-glycanohydrolase) to the hydrogel and incubating at 37° C. until the gel liquefies and collagenase for the degradation of collagen (or other enzymes for ECM degradation). The dissolved solution can then be collected and centrifuged. β-Agarase/Collagenase treatment has no effects on mammalian cell viability or functional compatibility (Carlsson J, Malmqvist M. Effects of bacterial agarase on agarose gel in cell culture. In Vitro [Internet]. 1977 [cited 2015 Nov. 2]; 13: 417-22. doi: 10.1007/BF02615101). The isolated invaded cell population can then be subjected to in-HMCA cycling procedure based on the invasion assay model. The cell pool can be suspended in fresh medium and re-seeded in an additional HMC for a second sequence of spheroid formation followed by an invasion assay. This cyclic strategy can create an enriched PrTMIC population. Following two to four rounds of reseeding and invasion, the collected cells can be subjected to further molecular phenotyping and functional characterization.

### Example 11

### Effect of Environmental Stiffness on Breast Cancer Multicellular 3D Structures, Formation and Growth In Vitro

Biological tissues normally possess varying levels of rigidity, which contribute to the performance of their physiological functions. Changes in tissue rigidity may reflect transformation from a normal to a pathological state. Cancer cells within the tumor are influenced by the mechanical conditions of their microenvironment, which can drive cell fate. Embodiments of the present HMC array can be used to mimic the desired surrounding rigidity in vitro for 3D breast cancer object/structure formation and growth. Non-adherent, non-tethered 3D objects were generated from single cells within a hydrogel array, cultured under various mechanical conditions which were created by procedure of agarose embedding, and measured at single-object resolution exploiting the advantageous mechanical and optical properties of agarose. This study demonstrates differences in the in vitro development of 3D breast cancer micro-tissues under various rigidity conditions. Individual 3D breast cancer structures revealed significant differences in object growth rate, morphology and vital features that are associated with the extent of environmental rigidity, the point in time at which embedding was performed and the initial number of seeded cells. The 3D objects initiated from less than six cells are significantly different from those initiated by more cells and demonstrate a growth rate independent from surrounding rigidity. Additionally, the control culture of 3D objects grown freely under low-rigidity conditions lacks the specific subset of the pre-invasive phenotype which developed in the stiffer surroundings.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A cell culturing device comprising:
a) a plate having at least one well with a through-hole formed at a bottom wall thereof; and
b) a hydrogel matrix disposed in said through hole.

2. The device of claim 1, further comprising:
c) at least one chamber formed in said hydrogel matrix.

3. The device of claim 1, wherein said through-hole is shaped so as to trap said hydrogel matrix therewithin.

4. The device of claim 1, wherein said hydrogel matrix extends into said at least one well.

5. The device of claim 1, further comprising an optically transparent support positioned under said plate, wherein said hydrogel matrix disposed in said through-hole contacts a top surface of said support.

6. The device of claim 1, wherein said through hole is shaped as a truncated cone.

7. The device of claim 3, wherein an inner surface of said through hole includes at least one undercut region.

8. The device of claim 3, wherein an inner surface of said through hole includes protrusions directed radially inward.

9. The device of claim 2, comprising a plurality of picoliter to microliter chambers formed in said hydrogel matrix.

10. The device of claim 1, further comprising a ring positionable in said at least one well, said ring including a circumferential inner groove.

11. The device of claim 1, further comprising a double ring insert positionable in said at least one well, said double ring insert including a central opening defined by an inner ring of said double ring insert and a plurality of compartments defined between said inner ring and an outer ring of said double ring insert.

12. A method of manufacturing a culturing device comprising:
a) providing a plate having at least one well with a through-hole formed at a bottom wall thereof;
b) filling said through-hole with a hydrogel; and
c) embossing at least one cell culturing chamber in said hydrogel.

13. The method of claim 12, further comprising positioning a double ring insert within said well prior to (b).

14. The method of claim 13, wherein said double ring insert includes a central opening defined by an inner ring of said double ring insert and a plurality of compartments defined between said inner ring and an outer ring of said double ring insert.

15. The method of claim 14, wherein said double ring insert includes at least one circumferential groove within an inner wall of said inner ring for trapping said hydrogel.

16. A cell culturing device comprising:
a) a plate having at least one well with a through-hole formed at a bottom wall thereof;
b) a hydrogel matrix disposed in said through-hole; and
c) a gel disposed on top of said hydrogel matrix.

17. The device of claim 16, wherein said gel includes at least one extracellular matrix (ECM) component.

18. (canceled)

19. The device of claim 16, wherein said gel including at least one extracellular matrix (ECM) component is disposed within a double ring insert positioned in said at least one well, said double ring insert including a central opening defined by an inner ring of said double ring insert and a plurality of compartments defined between said inner ring and an outer ring of said double ring insert.

20. (canceled)

21. A method of culturing one or more cell types comprising:
(a) providing the cell culturing device of claim 9;
(b) seeding one or more cell types within said picoliter to microliter chamber; and
(c) subjecting the cell culturing device to conditions suitable for culturing said one or more cell types.

22. The method of claim 21, wherein said chamber is formed within a plurality of compartments each being for seeding a cell type.
